(19) United States
(12) Patent Application Publication
(10) Pub. No.: US 2024/0177440 A1
(43) Pub. Date: May 30, 2024

(54) THREE-DIMENSIONAL INTRAORAL MODEL PROCESSING DEVICE AND THREE-DIMENSIONAL INTRAORAL MODEL PROCESSING METHOD

(71) Applicant: MEDIT CORP., Seoul (KR)

(72) Inventors: Sung Hoon LEE, Seoul (KR); Jin Young KIM, Seoul (KR)

(73) Assignee: MEDIT CORP., Seoul (KR)

(21) Appl. No.: 18/283,558

(22) PCT Filed: Mar. 22, 2022
(86) PCT No.: PCT/KR2022/003986

§ 371 Date: Sep. 22, 2023

(30) Foreign Application Priority Data
Mar. 24, 2021 (KR) 10-2021-0038280

(51) Int. Cl. G06T 19/20; G16H 50/50

(52) U.S. Cl. CPC G06T 19/20; G16H 50/50; G06T 2200/24; G06T 2210/41; G06T 2219/2004

(57) ABSTRACT

A three-dimensional intraoral model processing device and an operating method of the three-dimensional intraoral model processing device are provided. A three-dimensional intraoral model processing device is configured to identify a missing tooth included in scanned teeth of a three-dimensional intraoral model obtained by scanning teeth, generate a planned prosthetic tooth for prosthetizing the missing tooth by using at least one of the scanned teeth of the three-dimensional intraoral model, move the scanned teeth including the generated planned prosthetic tooth to a final target position, and display a target intraoral model including the scanned teeth moved to the final target position.

### TECHNICAL FIELD

Embodiments relate to a three-dimensional intraoral model processing device and a three-dimensional intraoral model processing method.

Specifically, embodiments relate to a three-dimensional intraoral model processing device and a three-dimensional intraoral model processing method that obtain a final position of a tooth for an orthodontic plan.

### BACKGROUND ART

There are various fields in dental treatment of patients. Orthodontic treatment is an example of the dental treatment.

In order to perform the orthodontic treatment, for example, an orthodontic device, such as a bracket, is installed on teeth of a patient, and a wire is connected to at least one installed bracket. Correction of a position of a tooth may be performed by moving at least one tooth to a target position, that is, a final position or a target position of the tooth by using a bracket connected to a wire.

Because orthodontic treatment is performed by an operation of moving teeth at an initial position of the teeth of a patient to a target position, it is important to accurately determine a final position or a target position of the teeth indicating to which position the teeth at the initial position are to be moved in an orthodontic plan.

### DISCLOSURE

### Technical Problem

The present disclosure provide a three-dimensional intraoral model processing method for obtaining a final position of a tooth to be moved for an orthodontic plan, and a device of performing an operation according thereto.

### Technical Solution

According to one embodiment, a three-dimensional intraoral model processing device includes a memory storing one or more instructions, and a processor, by executing the one or more instructions, being configured to identify a missing tooth in scanned teeth of a three-dimensional intraoral model obtained by scanning teeth, generate a planned prosthetic tooth for prosthetizing the missing tooth by using one of the scanned teeth of the three-dimensional intraoral model, move the scanned teeth including the generated planned prosthetic tooth to a final target position, and display a target intraoral model including the scanned teeth moved to the final target position.

According to one embodiment, the processor, by executing the one or more instructions, is configured to individualize the scanned teeth of the three-dimensional intraoral model, and assign a tooth number to each of the individualized scanned teeth, wherein a tooth number that is not assigned to a scanned tooth is identified as a tooth number of the missing tooth.

According to one embodiment, the processor, by executing the one or more instructions, is configured to generate the planned prosthetic tooth for prosthetizing the missing tooth according to a user input for selecting the tooth number of the missing tooth.

According to one embodiment, the processor, by executing the one or more instructions, is configured to generate closed teeth by combining each of the scanned teeth with a dental root of a template tooth of a corresponding tooth number, wherein a closed tooth corresponding to the planned prosthetic tooth is generated by using a scanned tooth symmetrical to the missing tooth.

According to one embodiment, the processor, by executing the one or more instructions, is configured to identify a position of a template tooth corresponding to a tooth number of the missing tooth, and generate the closed tooth corresponding to the planned prosthetic tooth by symmetrically moving and arranging a closed tooth symmetrical to the template tooth corresponding to the tooth number of the missing tooth to a position of the template tooth corresponding to the tooth number of the missing tooth.

According to one embodiment, the processor, by executing the one or more instructions, is configured to identify a position of a template tooth corresponding to a tooth number of the missing tooth, and generate the closed tooth corresponding to the planned prosthetic tooth by symmetrically moving the scanned tooth symmetrical to the missing tooth and combining the scanned tooth symmetrical to the missing tooth with a dental root of the template tooth corresponding to the tooth number of the missing tooth.

According to one embodiment, the processor, by executing the one or more instructions, is configured to obtain the scanned teeth moved to the final target position by aligning the closed teeth with a customized curve generated based on the scanned teeth.

According to one embodiment, the processor, by executing the one or more instructions, is configured to control transparency of a color of the closed tooth generated in correspondence with the tooth number of the missing tooth to be adjustably displayed.

According to one embodiment, the processor, by executing the one or more instructions, is configured to output a user interface including one or more items selectable by a user according to a user input for selecting a tooth number recognized as the missing tooth, and the one or more items may include at least one of a first item selectable to recognize a scanned tooth corresponding to a tooth number recognized as the missing tooth among the scanned teeth of the three-dimensional intraoral model, or a second item selectable to provide a planned prosthetic tooth for prosthetizing the recognized missing tooth.

According to one embodiment, the processor, by executing the one or more instructions, is configured to receive a user input designating a scanned tooth corresponding to the missing tooth according to a user input for selecting the first item, assign a tooth number to the designated scanned tooth, and output the assigned tooth number.

According to one embodiment, an operating method of a three-dimensional intraoral model processing device includes identifying a missing tooth among scanned teeth of a three-dimensional intraoral model obtained by scanning teeth, generating a planned prosthetic tooth for prosthetizing the missing tooth by using one of the scanned teeth of the three-dimensional intraoral model, moving the scanned teeth including the generated planned prosthetic tooth to a final target position, and displaying a target intraoral model including the scanned teeth moved to the final target position.

According to one embodiment, a non-transitory computer-readable storage medium in which a program including at least one instruction is recorded to perform a three-dimensional intraoral model processing method by using a computer is provided, and the three-dimensional intraoral model processing method includes identifying a missing tooth among scanned teeth of a three-dimensional intraoral model obtained by scanning teeth, generating a planned prosthetic tooth for prosthetizing the missing tooth by using one of the scanned teeth of the three-dimensional intraoral model, moving the scanned teeth including the generated planned prosthetic tooth to a final target position, and displaying a target intraoral model including the scanned teeth moved to the final target position.

### Advantageous Effects

A three-dimensional intraoral model processing method according to an embodiment and a device performing an operation according thereto obtain a final position to which a tooth is to be moved based on teeth of a patient in an orthodontic plan, and accordingly, the final position more suitable for a tooth arrangement state of the patient may be obtained.

### DESCRIPTION OF DRAWINGS

The present disclosure may be easily understood from the following detailed description and the accompanying drawings, wherein reference numerals denote constituent elements.

FIG. 1 illustrates an example of a three-dimensional intraoral model including a missing tooth, according to an embodiment.

FIG. 2 is a diagram illustrating a three-dimensional intraoral model processing system according to an embodiment.

FIG. 3 is a block diagram illustrating a data processing device according to an embodiment.

FIG. 4 is a flowchart illustrating a three-dimensional intraoral model processing method of a data processing device, according to an embodiment.

FIG. 5 is a reference view illustrating a method of individualizing teeth of a three-dimensional intraoral model by using a template tooth model, according to an embodiment.

FIG. 6 illustrates an example of a graphical user interface for receiving a user input for designating a planned prosthetic tooth for prosthetizing a missing tooth by a data processing device, according to an embodiment.

FIG. 7 illustrates an example of a graphical user interface that may be provided by a data processing device in relation to a missing tooth, according to an embodiment.

FIG. 8 illustrates an example of a graphical user interface that may be provided according to an input for selecting a missing tooth in a graphical user interface, according to an embodiment.

FIG. 9 illustrates another example of a graphical user interface that may be provided according to an input for selecting a missing tooth in a graphical user interface, according to an embodiment.

FIG. 10 illustrates another example of a graphical user interface that may be provided according to an input for selecting a missing tooth in a graphical user interface, according to an embodiment.

FIG. 11 is a flowchart illustrating processes of a method of generating and displaying a target intraoral model in which scanned teeth are moved to a target position, according to an embodiment.

FIG. 12 is a reference diagram illustrating a method of generating closed teeth of scanned teeth, according to an embodiment.

FIG. 13 is a reference diagram illustrating an example of a method of generating closed teeth of scanned teeth including a missing tooth and arranging the generated closed teeth in a template tooth model, according to an embodiment.

FIG. 14 is a reference view illustrating another example of a method of generating closed teeth of scanned teeth including a missing tooth and arranging the generated closed teeth in a template tooth model, according to an embodiment.

FIG. 15 is a reference view illustrating an example of a method of aligning scanned teeth aligned in a template tooth model with a customized curve, according to an embodiment.

FIG. 16 illustrates an example of a graphical user interface showing a current tooth position and a final tooth position of a patient, according to an embodiment.

### BEST MODE

### Mode for Invention

The present specification clarifies the scope of the present disclosure, describes the principles of the present disclosure, and discloses embodiments such that those skilled in the art to which the present disclosure belongs may practice the present disclosure. The embodiments may be implemented in various forms.

The same reference numbers designate the same components throughout the specification. The present specification does not describe all components of embodiments, and general descriptions or redundant descriptions between embodiments in the technical field to which the present disclosure belongs are omitted. A term ‘part or portion’ used in the specification may be implemented by software or hardware, and depending on embodiments, a plurality of ‘parts or portions’ may be implemented as one component (one unit or one element), or a single ‘part or portion’ may also include a plurality of units or elements. Hereinafter, operation principles and embodiments of the present disclosure are described with reference to the accompanying drawings.

In the present specification, an image may include at least one tooth or an image (hereinafter, referred to as an ‘intraoral image’) representing an oral cavity including at least one tooth.

Also, in the present specification, an image may indicate a two-dimensional image of an object, or a three-dimensional model or a three-dimensional image representing the object in a three dimension. Also, in the present specification, an image may refer to data required to represent an object in a two dimension or a three dimension, for example, raw data and so on obtained by at least one image sensor. Specifically, the raw data is data obtained to generate an intraoral image and may be data (for example, two-dimensional data) obtained by at least one image sensor included in an intraoral scanner when the inside of an oral cavity of a patient that is an object is scanned by using the intraoral scanner.

In the present specification, an ‘object’ may include a tooth, gingiva, at least a part of an oral cavity, and/or artificial structures (for example, orthodontic devices, implants, artificial teeth, orthodontic aids to be inserted into the oral cavity, or so on) that are insertable into the oral cavity, and/or so on. Here, the orthodontic device may include at least one of a bracket, an attachment, an orthodontic screw, a lingual orthodontic device, and a removable orthodontic retainer.

Hereinafter, embodiments will be described in detail with reference to the drawings.

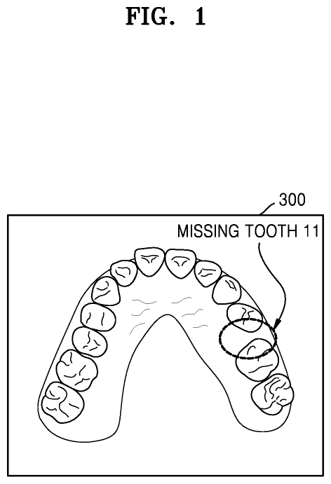

FIG. 1 illustrates an example of a three-dimensional intraoral model including a missing tooth, according to one embodiment.

In dental treatment, particularly orthodontic treatment, treatment for moving or rotating one or more teeth of a patient may be performed to straighten the teeth of the patient. For a plan of the orthodontic treatment, current teeth of the patient may be moved or rotated to a target position according to an orthodontic plan, and accordingly, a target intraoral model or a final intraoral model having an expected tooth arrangement state needs to be obtained. In addition, the target intraoral model also needs to be shown to the patient to show the patient undergoing an orthodontic treatment how much the tooth state of the patient changes according to the orthodontic treatment.

When the target intraoral model is generated based on a jaw arch of the patient, the target intraoral model may be obtained to be more natural to the patient. To this end, a dental state of the patient is used when the target intraoral model is generated, and in this case, a missing tooth may be included in teeth of the patient. When teeth do not exist inherently or are removed since birth, the missing tooth may indicate the corresponding missed tooth.

Referring to FIG. 1, a three-dimensional intraoral model 300 may include a missing tooth 11.

The three-dimensional intraoral model 300, which is generated based on two-dimensional image data obtained by scanning an oral cavity or a tooth model that is an object, may include one or more teeth and gingiva, and the one or more teeth may include a missing tooth 11. The missing tooth 11 indicates that a tooth which should normally be there is missing and lost, and accordingly, it is preferable that an orthodontic plan of a patient includes a prosthetic tooth for prosthetizing the missing tooth 11 and a target intraoral model is generated with the prosthetic tooth included therein. However, because the three-dimensional intraoral model 300 obtained by scanning teeth of the patient does not show the tooth that should be there, it is only possible to obtain a tooth number of the missing tooth 11, that is, which tooth is the missing tooth 11, but it is difficult to determine a position of the missing tooth 11, that is, a correct position of a planned prosthetic tooth for prosthetizing the missing tooth 11. Therefore, when a target intraoral model is generated in a state of the three-dimensional intraoral model 300 including the missing tooth 11 illustrated in FIG. 1, the target intraoral model may be generated inaccurately and unnaturally. Therefore, embodiments that provide a method and device capable of obtaining an accurate and natural target intraoral model even when the three-dimensional intraoral model includes a missing tooth will be described hereinafter.

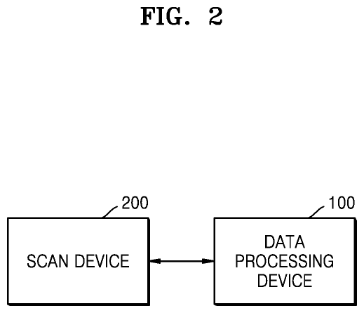

FIG. 2 is a diagram illustrating a three-dimensional intraoral model processing system according to an embodiment.

Referring to FIG. 2, the three-dimensional intraoral model processing system may include a scan device 200 and a data processing device 100.

The scan device 200 may scan an object, and the object may include any object or body to be scanned. For example, an object may include at least a part of a body of a patient including an oral cavity or a face, or include a tooth model. The scan device 200 may include a handheld scanner that scans an object held in a user's hand, a model scanner that scans an installed tooth model while moving around the installed tooth model, or so on.

For example, an intraoral scanner, which is a type of the handheld scanner, may obtain an image of an oral cavity including at least one tooth by being inserted into the oral cavity and scanning teeth in a non-contact manner. Also, the intraoral scanner may have a form capable of being drawn in and out of an oral cavity, and scans the inside of the oral cavity of a patient by using at least one image sensor (for example, an optical camera or so on). The intraoral scanner may obtain surface information of an object as raw data to capture an image of a surface of at least one of a tooth which is an object, gingiva, and an artificial structure (for example, an orthodontic device including a bracket and a wire, implant, an artificial tooth, orthodontic aids inserted into an oral cavity, or so on). The intraoral scanner is suitable for scanning an oral cavity as it is easily drawn in and out of the oral cavity, and may also scan a body part, such as a face of a patient.

The scan device 200 may obtain image data by using an optical triangulation method, a confocal method, or other methods.

The image data obtained by the scan device 200 may be transmitted to the data processing device 100 connected through a wired or wireless communication network. Of course, the scan device 200 may transmit the obtained image data to a cloud system instead of directly transmitting the obtained image data to the data processing device 100, and the data processing device 100 may receive image data through the cloud system.

The data processing device 100 may be implemented by any electronic device that is connected to the scan device 200 through a wired or wireless communication network, receives a two-dimensional image obtained by scanning an oral cavity from the scan device 200, and generates, processes, displays, and/or transmits an oral image based on the received two-dimensional image. Of course, the data processing device 100 may receive a two-dimensional image through a cloud system instead of directly receiving from the scan device 200.

The data processing device 100 may generate at least one of information generated by processing the two-dimensional image data and an intraoral image generated by processing the two-dimensional image data, based on the two-dimensional image data received from the scan device 200, and may display the generated information and intraoral image through a display.

The data processing device 100 may be implemented by a computing device, such as a smartphone, a laptop computer, a desktop computer, a PDA, or a tablet PC, but is not limited thereto.

Also, the data processing device 100 may be provided in the form of a server (or a server device) for processing intraoral images.

Also, the scan device 200 may transmit raw data obtained through scanning to the data processing device 100 as it is. In this case, the data processing device 100 may generate a three-dimensional intraoral image three-dimensionally representing an oral cavity based on the received raw data. Also, a ‘three-dimensional intraoral image’ may be generated by three-dimensionally modeling an internal structure of an oral cavity based on the received raw data, and may also be referred to as a ‘three-dimensional intraoral image’, a ‘digital intraoral model’, or a ‘three-dimensional intraoral model’. Hereinafter, a model or image representing two-dimensionally or three-dimensionally an oral cavity is collectively referred to as an ‘intraoral image’.

Also, the data processing device 100 may analyze, process, and display the generated intraoral image, and/or transmit the generated intraoral image to an external device.

In another example, the scan device 200 may obtain raw data through scanning, process the obtained raw data, generate an image corresponding to an oral cavity that is an object, and transmit the generated image to the data processing device 100. In this case, the data processing device 100 may analyze, process, display, and/or transmit the received image.

In the embodiment, the data processing device 100 may be an electronic device that may generate and display an intraoral image three-dimensionally representing an oral cavity including one or more teeth, which is described in detail below.

According to one embodiment, when receiving raw data obtained by scanning an oral cavity of a patient from the scan device 50, the data processing device 100 may process the received raw data to generate a three-dimensional intraoral model.

According to one embodiment, the data processing device 100 may perform processing of establishing an orthodontic plan for a patient by using a customized curve suitable for an oral cavity of the patient based on a three-dimensional intraoral model generated based on the oral cavity of the patient. The data processing device 100 may generate a target intraoral model by moving the scanned teeth included in the three-dimensional intraoral model generated based on an oral cavity of a patient to a target movement position after orthodontic treatment as one of orthodontic plan establishments, and may display the generated target intraoral model.

According to one embodiment, when a three-dimensional intraoral model of a patient includes a missing tooth, the data processing device 100 may generate a target intraoral model by arranging a planned prosthetic tooth at a position of the missing tooth by using other teeth or by moving the scanned teeth to a target position while securing a space for the planned prosthetic tooth. For example, in order to prepare the planned prosthetic tooth to be arranged at a position of a missing tooth, a tooth symmetrical to the position of the missing tooth may be used. The tooth in a symmetric relationship with the position of the missing tooth may include, for example, a tooth in a symmetrical position with the missing tooth, a tooth corresponding to a tooth number that is symmetric to a tooth number of the missing tooth, a template tooth corresponding to the tooth number of the missing tooth in a template tooth model, a template tooth symmetrical to the template tooth corresponding to the tooth number of the missing tooth in the template tooth model, and so on. In this way, after orthodontic treatment of the scanned teeth, a target intraoral model may be generated more naturally by arranging a planned prosthetic tooth corresponding to the missing tooth or securing a space where the planned prosthetic tooth is arranged, when a three-dimensional intraoral model includes a missing tooth.

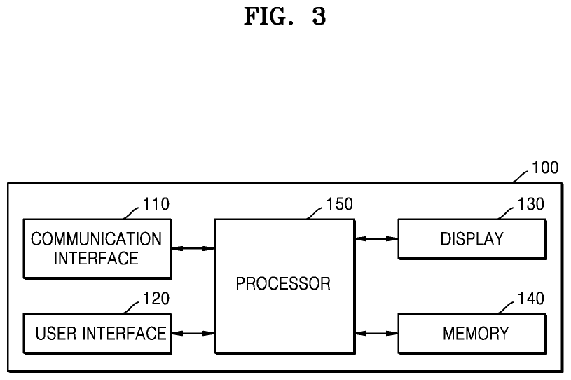

FIG. 3 is a block diagram illustrating the data processing device 100 according to the embodiment.

Referring to FIG. 3, the data processing device 100 may include a communication interface 110, a user interface 120, a display 130, a memory 140, and a processor 150.

The communication interface 110 may communicate with at least one external electronic device through a wired or wireless communication network. Specifically, the communication interface 110 may communicate with the intraoral scanner under control by the processor 160. The communication interface 110 may communicate with an external electronic device, a server, or so on connected through a wired or wireless communication network under control by a processor.

The communication interface 110 may communicate with an external electronic device (for example, an intraoral scanner, a server, an external medical device, or so on) through a wired or wireless communication network. Specifically, the communication interface 110 may include at least one short-range communication module that performs communication according to a communication standard, such as Bluetooth, Wi-Fi, Bluetooth low energy (BLE), near field communication (NFC)/radio frequency identification (RFID), Wi-Fi direct, ultra-wide band (UWB), or ZIGBEE.

Also, the communication interface 110 may further include a remote communication module that communicates with a server for supporting remote communication according to a telecommunication standard. Specifically, the communication interface 110 may include a remote communication module that performs communication through a network for Internet communication. Also, the communication interface 110 may include a remote communication module that performs communication through a communication network according to a communication standard, such as third generation (3G), fourth generation (4G), and/or fifth generation (5G).

Also, the communication interface 110 may include at least one port for being connected to an external electronic device (for example, an intraoral scanner or so on) through a wired cable to perform wired communication with the external electronic device. Accordingly, the communication interface 110 may perform communication with an external electronic device wired through at least one port.

The user interface 120 may receive a user input for controlling the data processing device 100. The user interface 120 may include a touch panel that detects a user's touch, a button that receives a user's push operation, and a user input device including a mouse, a keyboard, or so on for designating or selecting one point on a user interface screen.

Also, the user interface 120 may include a voice recognition device for voice recognition. For example, the voice recognition device may be a microphone, and the voice recognition device may receive a user's voice command or voice request. Accordingly, the processor 150 may control an operation corresponding to the voice command or voice request to be performed.

The display 130 displays a screen. Specifically, the display 130 may display a preset screen under control by the processor 160. Specifically, the display 130 may display a user interface screen including an intraoral image generated based on data obtained by scanning an oral cavity of a patient by using an intraoral scanner. Alternatively, the display 130 may display a user interface screen including information of a patient's dental treatment.

The memory 140 may store at least one instruction. Also, the memory 140 may store at least one instruction executed by the processor 150. Also, the memory 140 may store at least one program executed by the processor 150. Also, the memory 140 may store data (for example, raw data obtained through intraoral scan) received from an intraoral scanner. Alternatively, the memory 140 may store an intraoral image three-dimensionally representing an oral cavity. According to one embodiment, the memory 140 may include one or more instructions for obtaining target positions of teeth of an intraoral image from an orthodontic plan. According to one embodiment, the memory 140 may include one or more instructions for performing the method disclosed in the present disclosure to obtain final positions of teeth of an intraoral image.

The processor 150 may execute at least one instruction stored in the memory 140 to control an intended operation to be performed. Here, at least one instruction may be stored in an internal memory included in the processor 150 or stored in the memory 140 included in the data processing device 100 separately from the processor 150.

Specifically, the processor 150 may control at least one component included in the data processing device 100 such that an intended operation is performed by executing at least one instruction. Therefore, even when description is generated by using a case where a processor performs certain operations as an example, this may mean that the processor controls at least one component included in a data processing device to perform certain operations.

According to one embodiment, the processor 150 identifies a missing tooth among the scanned teeth of a three-dimensional intraoral model obtained by scanning teeth by executing one or more instructions stored in the memory 140, generates a planned prosthetic tooth for prosthetizing the missing tooth by using one of the scanned teeth of the three-dimensional intraoral model, moves the scanned teeth including the generated planned prosthetic tooth to final target positions, and displays a target intraoral model including the scanned teeth moved to the final target positions. Making of the planned prosthetic tooth for prosthetizing the missing tooth may be performed according to a user input for selecting a tooth number of the missing tooth.

According to one embodiment, the processor 150 may output a user interface including one or more items selectable by a user according to a user input for selecting a tooth number recognized as the missing tooth by executing one or more instructions stored in the memory 140, and the one or more items may include at least one of a first item selectable to recognize a scanned tooth corresponding to a tooth number recognized as the missing tooth among the scanned teeth of the three-dimensional intraoral model, and a second item selectable to provide a planned prosthetic tooth for prosthetizing the recognized missing tooth.

According to one embodiment, the processor 150 may execute one or more instructions stored in the memory 140 to receive a user input designating a scanned tooth corresponding to the missing tooth according to a user input for selecting the first item, to assign a tooth number to the designated scanned tooth, and to output the assigned tooth number.

According to one embodiment, the processor 150 may execute one or more instructions stored in the memory 140 to individualize the scanned teeth of the three-dimensional intraoral model and to assign a tooth number to each of the individualized scanned teeth and may identify a tooth number not assigned to the scanned tooth as a tooth number of the missing tooth.

According to one embodiment, the processor 150 may execute one or more instructions stored in the memory 140 to generate a closed tooth by combining each of the scanned teeth with a dental root of a template tooth having a corresponding tooth number, and may generate a closed tooth corresponding to the planned prosthetic tooth by using the scanned tooth symmetrical to the missing tooth.

According to one embodiment, the processor 150 may identify a position of a template tooth corresponding to a tooth number of the missing tooth by executing one or more instructions stored in the memory 140, and may generate a closed tooth corresponding to the planned prosthetic tooth by symmetrically moving and arranging the closed tooth symmetrical to the template tooth corresponding to a tooth number of the missing tooth to the position of the template tooth corresponding to the tooth number of the missing tooth.

According to one embodiment, the processor 150 may identify a position of a template tooth corresponding to a tooth number of the missing tooth by executing one or more instructions stored in the memory 140, and may generate a closed tooth corresponding to the planned prosthetic tooth by symmetrically moving a scanned tooth symmetrical to the missing tooth and combining the scanned tooth with a dental root of a template tooth corresponding to a tooth number of the missing tooth.

According to one embodiment, the processor 150 may align the closed teeth with a customized curve generated based on the scanned teeth by executing one or more instructions stored in the memory 140, and accordingly, the scanned teeth moved to the final target positions may be obtained.

According to one embodiment, the processor 150 may adjust transparency of a color of a closed tooth generated in correspondence with a tooth number of the missing tooth so as to be displayed, by executing one or more instructions stored in the memory 140.

According to one example, the processor 150 may be implemented in a form including at least one internal processor and a memory device (for example, random access memory (RAM), read only memory (ROM), or so on) for storing at least one of a program, an instruction, a signals, and data to be processed or used by the internal processor.

Also, the processor 150 may include a graphics processing unit (GPU) for graphics processing corresponding to video. Also, the processor 150 may be implemented as a system on chip (SoC) in which a core is integrated with a GPU. Also, the processor 150 may include multiple cores more than or equal to a single core. For example, the processor 150 may include dual cores, triple cores, quad cores, hexa cores, octa cores, deca cores, dodeca cores, hexadecimal cores, or so on.

In the embodiment, the processor 150 may generate an intraoral image based on a two-dimensional image received from an intraoral scanner.

Specifically, under control by the processor 150, the communication interface 110 may receive data obtained by an intraoral scanner, for example, raw data obtained through intraoral scanning. Also, the processor 150 may generate a three-dimensional intraoral image three-dimensionally representing an oral cavity based on the raw data received from the communication interface 110. For example, the intraoral scanner may include an L camera corresponding to a left field of view and an R camera corresponding to a right field of view to recover a three-dimensional image according to an optical triangulation method. In addition, the intraoral scanner may obtain L image data corresponding to the left field of view and R image data corresponding to the right field of view respectively from the L camera and the R camera. Subsequently, the intraoral scanner (not illustrated) may transmit raw data including the L image data and R image data to the communication interface 110 of the data processing device 100.

Then, the communication interface 110 may transmit the received raw data to the processor 150, and the processor 150 may generate an intraoral image three-dimensionally representing an oral cavity based on the received raw data.

Also, the processor 150 may control the communication interface 110 to directly receive an intraoral image three-dimensionally representing an oral cavity from an external server, a medical device, or so on. In this case, the processor 150 may obtain a three-dimensional intraoral image without generating a three-dimensional intraoral image based on the raw data.

According to the embodiment, performing, by the processor 150, operations, such as ‘extraction’, ‘obtainment’, and ‘generation’ may include that the processor 160 executes at least one instruction to directly perform the above-described operations and controls other components to perform the above-described operations.

In order to implement the embodiments of the present disclosure, the data processing device 100 may include only some of the components illustrated in FIG. 3 or may include more components than the components illustrated in FIG. 3.

Also, the data processing device 100 may store and execute dedicated software linked to an intraoral scanner. Here, the dedicated software may be referred to as a dedicated program, a dedicated tool, or a dedicated application. When the data processing device 100 operates in conjunction with an intraoral scanner, the dedicated software stored in the data processing device 100 may be connected to the intraoral scanner to receive data obtained through intraoral scanning in real time. For example, a product i500 which is an intraoral scanner of the Medit includes dedicated software for processing data obtained through intraoral scanning. Specifically, the Medit produces and distributes ‘Medit Link’, that is software for processing, managing, using, and/or transmitting data obtained by an intraoral scanner (for example, i500). Here, ‘dedicated software’ indicates a program, a tool, or an application that may operate in conjunction with an intraoral scanner, and accordingly, various intraoral scanners developed and sold by various manufacturers may be used in common. Also, the dedicated software may be produced and distributed separately from an intraoral scanner for performing the intraoral scan.

The data processing device 100 may store and execute the dedicated software corresponding to the product i500. The transmission software may perform at least one operation for obtaining, processing, storing, and/or transmitting an intraoral image. Here, the dedicated software may be stored in a processor. Also, the dedicated software may provide a user interface for use of data obtained by an intraoral scanner. Here, a user interface screen provided by the dedicated software may include an intraoral image generated according to the embodiment described above.

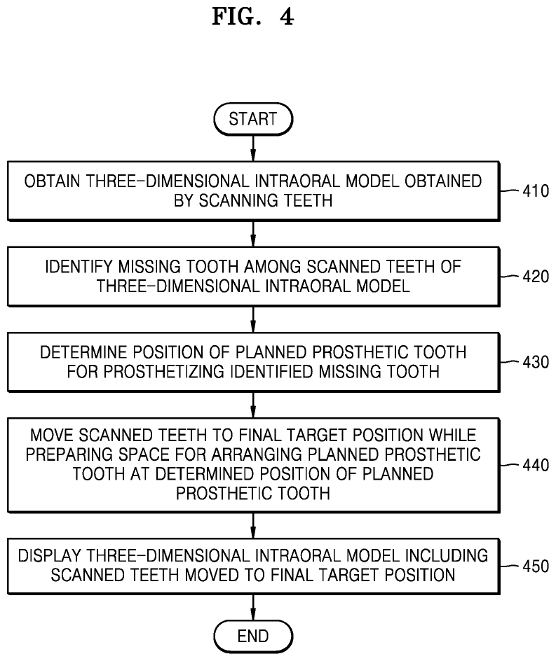

FIG. 4 is a flowchart illustrating a three-dimensional intraoral model processing method of a data processing device, according to an embodiment.

Referring to FIG. 4, in operation 410, the data processing device 100 may obtain a three-dimensional intraoral model generated by scanning teeth.

According to one embodiment, the data processing device 100 may receive two-dimensional data generated by scanning teeth from the scan device 100 as illustrated in FIG. 2 and generate a three-dimensional intraoral model based on the received two-dimensional data. Alternatively, according to one embodiment, the data processing device 100 may receive, from the scan device 100, the three-dimensional intraoral model generated based on two-dimensional data obtained by scanning teeth. Alternatively, according to one embodiment, the data processing device 100 may obtain a three-dimensional intraoral model stored in a memory.

In operation 420, the data processing device 100 may identify a missing tooth among the scanned teeth of the obtained three-dimensional intraoral model. In order to identify a missing tooth among the scanned teeth of the three-dimensional intraoral model, the data processing device 100 may first individualize the scanned teeth of the three-dimensional intraoral model and identify a missing tooth among the individualized scanned teeth.

Individualizing the scanned teeth may mean obtaining information of each of the scanned teeth included in the three-dimensional intraoral model. Individualization may also be called segmentation. Information of each tooth may include information of a shape of each tooth, information of a position of each tooth, and information of the number of each tooth.

According to one embodiment, the data processing device 100 may individualize teeth of the three-dimensional intraoral model by using a tooth model template. The tooth model template is standard data in which teeth have an ideal shape and are arranged in an ideal position, and the data processing device 100 may individualize the teeth of the three-dimensional intraoral model by aligning the teeth of the three-dimensional intraoral model with the tooth model template.

According to one embodiment, the data processing device 100 may also individualize the teeth of the three-dimensional intraoral model by using a neural network using artificial intelligence.

According to one embodiment, the data processing device 100 may obtain a tooth number corresponding to each scanned tooth by individualizing the scanned teeth of the three-dimensional intraoral model, and may recognize a tooth to which a tooth number is not assigned as a missing tooth.

According to one embodiment, the data processing device 100 may display each scanned tooth of the three-dimensional intraoral model and a tooth number corresponding to each scanned tooth. In this case, the data processing device 100 may display a tooth number of a tooth to which the tooth number is not assigned as a tooth number of the missing tooth.

According to one embodiment, the data processing device 100 may identify the missing tooth by receiving a user input designating the missing tooth. Specifically, the data processing device 100 may display a three-dimensional intraoral model on a display and identify a missing tooth by receiving a user input causing a user to designate the missing tooth among teeth of the displayed three-dimensional intraoral model.

According to one embodiment, the data processing device 100 may automatically identify a missing tooth. For example, the data processing device 100 may identify a missing tooth by using a neural network using artificial intelligence. Alternatively, for example, the data processing device 100 may identify a tooth that satisfies a certain condition as a missing tooth. For example, when a possibility or probability that any one of teeth of a three-dimensional intraoral model matches a certain tooth number is less than a threshold, the data processing device 100 may identify the tooth as a missing tooth.

In operation 430, the data processing device 100 may determine a position of a planned prosthetic tooth for prosthetizing the identified missing tooth.

According to one embodiment, the data processing device 100 may determine the position of the planned prosthetic tooth for prosthetizing the missing tooth as a position of a template tooth corresponding to a tooth number of the missing tooth. Specifically, the data processing device 100 may determine the position of the planned prosthetic tooth for prosthetizing the missing tooth as a central position of the template tooth corresponding to the tooth number of the missing tooth. The center position of the template tooth corresponding to the tooth number of the missing tooth may represent central coordinates of a bounding box surrounding the template tooth corresponding to the tooth number of the missing tooth. For example, when the tooth number of the missing tooth is 24, the data processing device 100 may use the central coordinates of the template tooth of the tooth number 24 among the template teeth of a template tooth model.

In operation 440, the data processing device 100 may move the scanned teeth of the three-dimensional intraoral model to a final target position while preparing a space for arranging the planned prosthetic tooth at the determined position of the planned prosthetic tooth.

According to one embodiment, in order to move the scanned teeth of the three-dimensional intraoral model to the final target position, the data processing device 100 may first move the scanned teeth of the three-dimensional intraoral model to a position of the template tooth, which has the corresponding tooth number, in the template tooth model. Then, the data processing device 100 may move the scanned teeth arranged at the positions of the template teeth of the template teeth model to be aligned with the customized curve generated based on a three-dimensional intraoral model of a patient. A position where the respective scanned teeth are aligned with the customized curve may be the final target position of the scanned teeth.

According to one embodiment, the data processing device 100 may generate a closed tooth by combining a dental root portion of the template tooth with the scanned tooth of the three-dimensional intraoral model to facilitate and realistically move the scanned tooth.

According to one embodiment, when moving the scanned teeth to positions of the template teeth of the template tooth model, the data processing device 100 may move the scanned teeth while preparing a space for arranging the planned prosthetic tooth at a position of the planned prosthetic tooth.

According to one embodiment, when moving the scanned teeth to the positions of the template teeth of the template tooth model, the data processing device 100 may move the scanned teeth while arranging the planned prosthetic teeth at the position of the planned prosthetic teeth.

According to one embodiment, when moving the scanned teeth to the positions of the template teeth of the template tooth model, the data processing device 100 may generate a closed tooth by combining a dental root portion of the template tooth with the scanned tooth and move the closed tooth may the generated closed tooth to the position of the template tooth of the template tooth model.

According to one embodiment, the data processing device 100 may generate the closed tooth of the planned prosthetic tooth by using the scanned tooth and the template tooth symmetrical to the missing tooth. For example, the data processing device 100 may generate the closed tooth by using a scanned tooth symmetrical to the missing tooth and a template tooth symmetrical to the missing tooth, and generate a closed tooth of the planned prosthetic tooth by mirroring the generated closed tooth, that is, performing reflection processing. For example, the data processing device 100 may generate a closed tooth by using a scanned tooth symmetrical to a missing tooth and a template tooth having a tooth number corresponding to a tooth number of the missing tooth.

According to one embodiment, when a tooth symmetrical to a missing tooth also corresponds to the missing tooth, the data processing device 100 may use a template tooth corresponding to a tooth number of the missing tooth.

According to one embodiment, the data processing device 100 may generate a customized curve based on one or more teeth included in a three-dimensional intraoral model. The “customized curve” may be referred to as such in the sense that a curve is generated based on one or more teeth included in a three-dimensional intraoral model of a patient.

According to one embodiment, the data processing device 100 may generate a customized arch line based on teeth at a predetermined position among teeth included in a three-dimensional intraoral model. The predetermined position may be, for example, a position of a tooth having the least movement. When the teeth in the predetermined position are lost, adjacent teeth may be used.

According to one embodiment, the data processing device 100 may obtain a final target position of a tooth by arranging the scanned teeth aligned with the position of each template tooth of the template tooth model to be aligned with the customized curve.

In operation 450, the data processing device 100 may display, on the display 130, the three-dimensional intraoral model including the scanned teeth moved to the final target position.

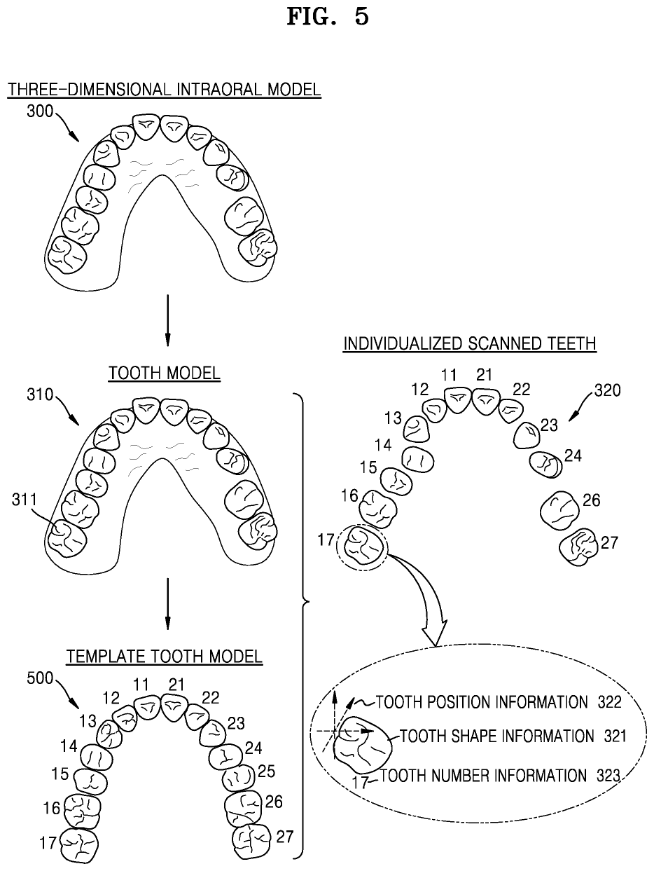

The three-dimensional intraoral model processing method according to the embodiment is described in detail with reference to FIGS. 5 to 16 below.

FIG. 5 is a reference view illustrating a method of individualizing teeth of a three-dimensional intraoral model using a template tooth model, according to one embodiment.

A three-dimensional intraoral model 300 may represent an image obtained by scanning an oral cavity of a patient.

A template tooth model 500 may represent three-dimensional tooth model data representing the most ideal dentition. The template tooth model 500 is tooth data in which each tooth has an ideal shape and teeth have an ideal arrangement, and a tooth number is attached to each tooth of the template tooth model 500. The template tooth model 500 may include shape data for each tooth, position data for each tooth, and a tooth number of each tooth. Referring to FIG. 5, the template tooth model 500 is composed of 14 teeth, and a numbering method for each tooth may change, and for example, in FIG. 5, the teeth are sequentially numbered in ascending order from a central tooth. That is, in FIG. 5, the teeth to the left of the center are numbered from tooth number 11 to the tooth number 17 from the center to the left, and the teeth to the right of the center are numbered from tooth number 21 to the tooth number 27 from the center to the right. A method of numbering teeth in this way is an example, and the method of numbering teeth may be determined in various ways.

The data processing device 100 may obtain a tooth model 310 by separating teeth and gingiva of the three-dimensional intraoral model 300 based on a curvature, thereby separating a tooth region. In addition, by aligning the template tooth model 500 with the tooth model 310, a number may be assigned to each tooth of the tooth model 310. When the data processing device 100 aligns the template tooth model 500 with the tooth model 310, various alignment algorithms may be used, for example, an algorithm, such as iterative closest point (ICP). The ICP is an algorithm for reducing between two point clouds and is an algorithm used to reconstruct a two-dimensional or three-dimensional surface from different scan data. The ICP algorithm fixes a point cloud which is called a reference, and transforms the point cloud, which is called a source, to best match the reference. The ICP algorithm may align a three-dimensional model by repeatably modifying transformation (a combination of translation and rotation) necessary to reducing an error metric representing a distance from the source to the reference. Various algorithms in addition to ICP may be used as an alignment algorithm, and for example, a Kabsch algorithm may be used therefor.

When the data processing device 100 aligns the template tooth model 500 with the tooth model 310 by using an ICP algorithm, the point cloud corresponding to the tooth model 310 may be the reference, and the point cloud corresponding to tooth model data may be the source.

When finding a tooth having a shape closest to a tooth 311, which is the first tooth in the tooth model 310, from the template tooth model 500, the data processing device 100 may determine that a tooth corresponding to a tooth number 17 of the tooth model template 500 is a tooth having a shape closest to the tooth 311 of the tooth model 310. In this way, tooth numbers may be obtained by finding the closest tooth for each tooth of the tooth model 310 from the template tooth model 500.

The data processing device 100 may obtain individualized tooth data 320 having information for each tooth by individualizing teeth of the tooth model 310 by using the template tooth model 500 as described above. The individualized scanned teeth 320 may include shape information 321, position information 322, and tooth number information 323 of each tooth included in the tooth model 310.

As illustrated in FIG. 5, the data processing device 100 may assign tooth numbers to respective scanned teeth while individualizing the scanned teeth of a three-dimensional intraoral model by using tooth numbers of a template tooth model, and when the three-dimensional intraoral model includes a missing teeth, a template tooth corresponding to the missing tooth may not be found, and accordingly, there may be a tooth number that is not assigned. Referring to FIG. 5, a tooth number 25 in the individualized scanned teeth is not assigned to any scanned tooth. Therefore, the data processing device 100 may recognize that the tooth number 25 corresponds to a missing tooth.

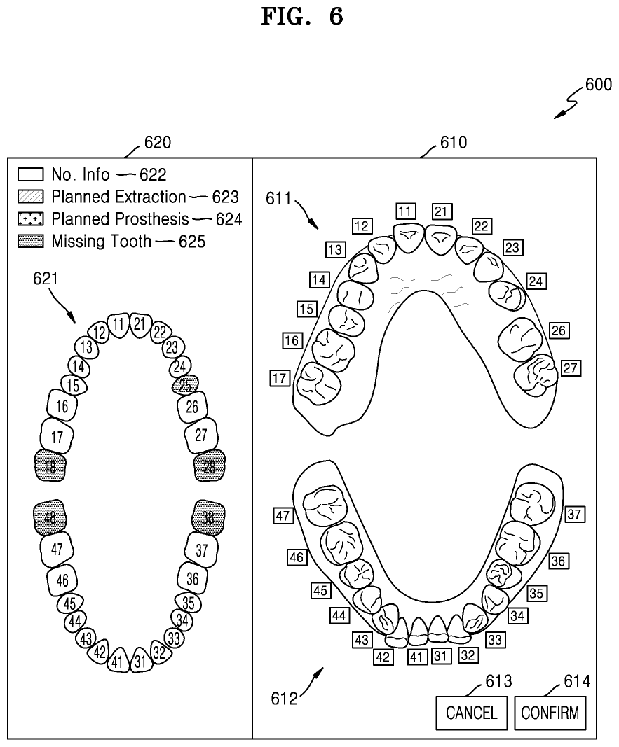

FIG. 6 illustrates an example of a graphical user interface for receiving a user input for designating a planned prosthetic tooth for prosthetizing a missing tooth by a data processing device, according to one embodiment.

Referring to FIG. 6, the data processing device 100 may display a graphical user interface 600 that may designate a planned prosthetic tooth on a display.

The graphical user interface 600 may include a first region 610 that displays a three-dimensional intraoral model obtained by scanning teeth of a patient, and a second region 620 that may display the state of the scanned teeth included in the three-dimensional intraoral model displayed in the first region 610 or receives an input for designating the state of the scanned teeth included in the three-dimensional intraoral model displayed in the first region 610.

The first region 610 includes upper jaw teeth 611 and lower jaw teeth 612 of a three-dimensional intraoral model representing teeth of a patient, and each tooth is numbered. Referring to FIG. 6, the upper jaw teeth 611 are numbered from a tooth number 11 to a tooth number 17 on the left side of the center, and are numbered from a tooth number 21 to a tooth number 27 on the right side of the center. Also, the lower jaw teeth 612 are numbered from a tooth number 41 to a tooth number 47 on the left side of the center, and are numbered from a tooth number 31 to a tooth number 37 on the right side of the center. However, a tooth corresponding to a tooth number 25 is not displayed in the upper jaw teeth 611, and accordingly, the tooth number 25 may indicate a tooth number of a missing tooth.

The second region 620 is a region for illustrating states of the scanned teeth of a three-dimensional intraoral model displayed in the first region 100, and for example, an image 621 of teeth numbered based on a template tooth model may be displayed. For example, the states of the scanned teeth may include states of no information 622, planned extraction 623, planned prosthesis 614, and missing tooth 625. The “no information” 622 indicates a state in which there is no information of a tooth, the “planned extraction” 623 indicates a tooth scheduled to be extracted, the “planned prosthesis” 624 indicates a tooth scheduled to be prosthetized, and the “missing tooth”” 625 indicates a state in which a tooth is missing and does not exist. The data processing device 100 may display each tooth corresponding to each tooth number in different colors, that is, display the no info, the planned extraction, the planned prosthesis, the missing tooth to be distinguished from each other.

Compared to the teeth of the template tooth model, the scanned teeth corresponding to tooth numbers 18, 25, 28, 38, and 48 in the three-dimensional intraoral model displayed in the first region 610 are missing, and accordingly, the teeth of the tooth numbers 18, 25, 28, 38, and 48 are displayed as missing teeth.

According to a user input received through the first region 610, the data processing device 100 may change states of teeth and display the changed states. For example, the data processing device 100 may change a missing tooth state to a planned prosthetic tooth state according to a user input for changing a missing tooth to a planned prosthetic tooth and display the planned prosthetic tooth state. For example, a user may check that a tooth number 25 is a missing tooth in the image 611 displayed in the second region 610 and may provide an input for changing the missing tooth to a planned prosthetic tooth state, and the data processing device 100 may change a state of the tooth number 25 from a missing tooth state to a planned prosthetic tooth state according to the input. For example, teeth of the tooth numbers 18, 28, 38, and 48 usually correspond to wisdom teeth even in the same missing tooth state, or no state change is input to the teeth of the tooth numbers 18, 28, 38, and 48 because it is determined that there is little need to install prosthetic teeth, and accordingly, the teeth of the tooth numbers 18, 28, 38, and 48 may be left as the missing tooth state.

However, the graphical user interface for designating a target tooth for prosthesis illustrated in FIG. 6 is only an example, and any type of interface may be used as long as the interface may receive a user input for designating a target tooth for prosthesis.

Receiving a user input through a user interface to designate a planned prosthetic tooth is an example, and the data processing device 100 may be implemented to automatically determine a planned prosthetic tooth in a three-dimensional intraoral model by using a neural network obtained by learning a method of determining a planned prosthetic tooth. For example, a neural network for determining a planned prosthetic tooth may be obtained by learning whether it is appropriate to arrange the planned prosthetic tooth at a position of a missing tooth based on the tooth number assigned to each scanned tooth of a three-dimensional intraoral model of a patient and a state of a tooth. In this case, when the three-dimensional intraoral model is generated, the data processing device 100 may automatically determine a planned prosthetic tooth from the generated three-dimensional intraoral model by using the neural network for determining the planned prosthetic tooth described above.

In this way, when a missing tooth is included in the scanned teeth of the three-dimensional intraoral model, and when a target intraoral model is generated by moving to a target position based on the scanned teeth including the missing tooth without any operation for the missing tooth, there is a large difference with the target intraoral model after an actual prosthetic tooth is generated later, and it is difficult to obtain a natural target intraoral model. Therefore, when the missing tooth included in the scanned teeth of a patient is scheduled for prosthesis, the data processing device 100 may generate a natural target intraoral model in consideration of a planned prosthetic tooth as a user automatically designates a planned prosthetic tooth corresponding to the missing tooth.

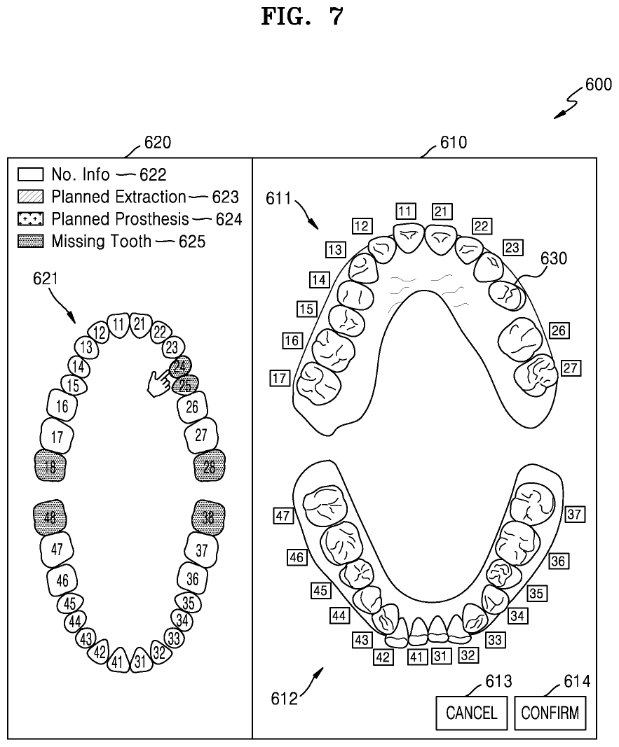

FIG. 7 illustrates an example of a graphical user interface provided by a data processing device in relation to a missing tooth, according to one embodiment.

The data processing device 100 may recognize a missing tooth due to various causes, although there is no missing tooth in the scanned teeth of a three-dimensional intraoral model, that is, there are teeth. Although there may be various causes, for example, when a shape of a tooth is significantly different from a shape of a template tooth or when a position of a tooth is significantly different from a position of a template tooth, the data processing device 100 may not recognize a tooth that significantly deviate from characteristics of a normal tooth and may recognize the tooth as a missing tooth.

The graphical user interface illustrated in FIG. 7 is mostly similar to the graphical user interface illustrated in FIG. 6, and a difference from the graphical user interface illustrated in FIG. 6 is that both a tooth number 24 and a tooth number 25 between a tooth number 23 and a tooth number 26 in a first region 610 are not displayed, and both the tooth number 24 and the tooth number 25 in the second region 620 are displayed as missing tooths. Actually, a tooth 630 is placed between a scanned tooth of the tooth number 23 and a scanned tooth of the tooth number 26 in the first region 610, and accordingly, the tooth 630 may be a tooth of the tooth number 24 or the tooth number 25. However, the data processing device 100 recognizes both the tooth number 24 and the tooth number 25 as missing teeth, and accordingly, it may be preferable to allow a user input to assign a tooth number of the tooth 630 therebetween.

In this way, an input for changing a corresponding missing tooth to a planned prosthetic tooth state as illustrated in FIG. 6 and an input enabling the corresponding missing tooth to be recognized as an existing tooth state as in the example of FIG. 7 may coexist with each other for the missing tooth. Therefore, the data processing device 100 may provide a menu for selecting one of two states corresponding to the missing tooth.

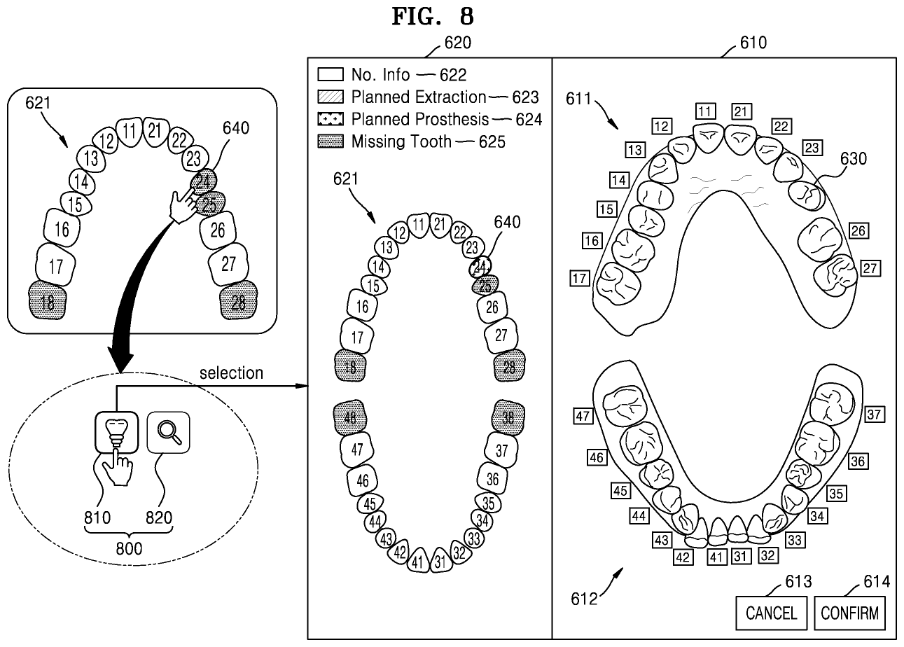

FIG. 8 illustrates an example of a graphical user interface that may be provided according to an input for selecting a missing tooth in a graphical user interface, according to one embodiment.

Referring to FIG. 8, when a user input for selecting a tooth number 24 displayed as a missing tooth state from an image 621 of a second region 620 is received by a user, the data processing device 100 may output a menu 800 in response to the user input. The menu 800 may include a first icon 810 for receiving a user input designating a missing tooth as a planned prosthetic tooth, and a second icon 820 for receiving a user input for recognizing a tooth corresponding to a tooth number of the missing tooth among scanned teeth displayed in a first region 610 to assign a tooth number to the recognized tooth.

For example, when a user selects the first icon 810 from the menu 800 displayed after selecting an image of a tooth number 24 representing a missing tooth state, the data processing device 100 may designate the tooth number 24 as a planned prosthetic tooth. The data processing device 100 may change a state of the tooth number 24 to a state 640 of a planned prosthetic tooth in a second region 620 and display the changed state.

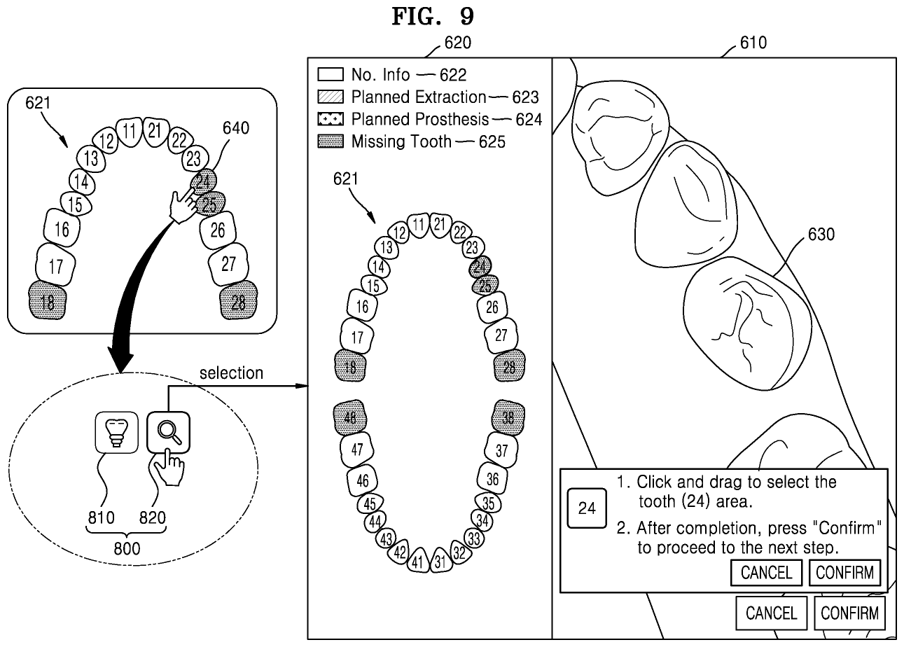

For example, when a user selects the second icon 820 from the menu 800, the data processing device 100 may assign a tooth number to a tooth 630 to which a tooth number is not assigned in the first region 610. This is described with reference to FIG. 9.

FIG. 9 illustrates another embodiment of a graphical user interface that may be provided according to an input for selecting a missing tooth in a graphical user interface, according to one embodiment.

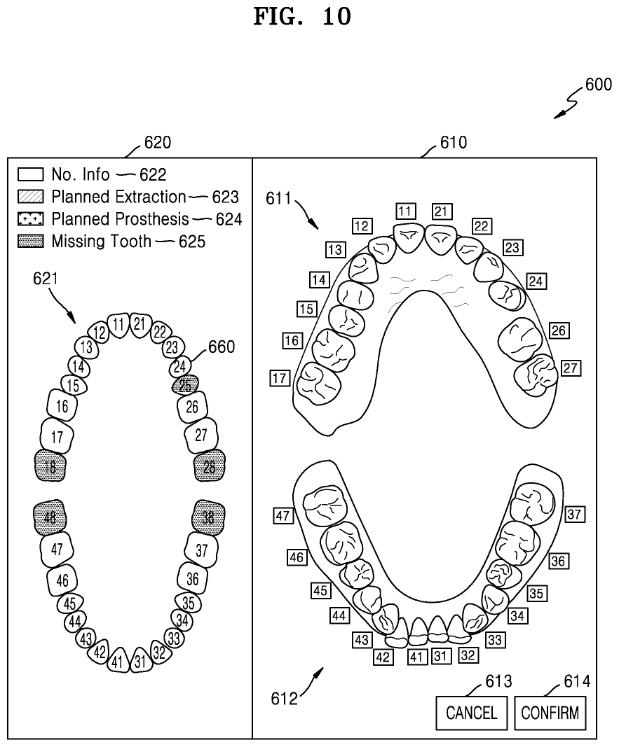

For example, when a user selects a second icon 820 from a menu 800, the data processing device 100 may assign a tooth number to a tooth 630 to which a tooth number is not assigned in a first region 610. More specifically, when a user selects the second icon 820 from the menu 800, the data processing device 100 may output a message 650 prompting the user to select the tooth number 24 from the first region 610. When a user clicks a tooth, to be associated with the tooth number 24 in the first region 610, that is, the tooth 630, in response to the message 650, the data processing device 100 may assign the tooth number 24 to the tooth 630. Also, the data processing device 100 may change a state of the tooth number 24 in the second region 620 and display the changed state. An example of the graphical user interface output according to the user input is illustrated in FIG. 10.

FIG. 10 illustrates another example of a graphical user interface that may be provided according to an input for selecting a missing tooth in a graphical user interface, according to one embodiment.

Referring to FIG. 10, for example, when a user selects a second icon 820 from a menu 800, the data processing device 100 may assign 24 to a tooth number of a tooth 630, to which a tooth number is not assigned, in a first region 610, and thereby, output the tooth number 24 assigned to the tooth 630. Also, the data processing device 100 may change a state of the tooth number 24 in the second region 620 from a missing tooth state to a no-information state 660 and display the changed state.

In this way, when selection of a confirmation menu 614 is received from a user after operations of recognizing a missing tooth or so on from the scanned teeth to assign a tooth number or designating the missing tooth as a planned prosthetic tooth by a user, the data processing device 100 may perform an operation of generating a target intraoral model, in which the scanned teeth are moved to a final target position, by reflecting states of the scanned teeth according to a user input.

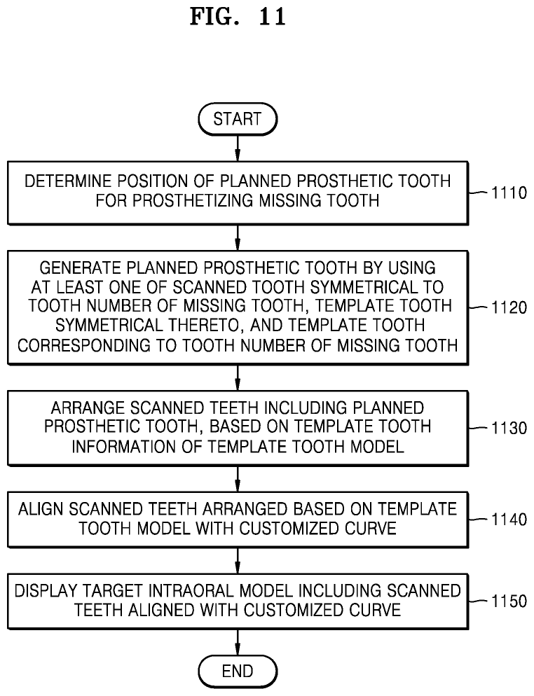

Hereinafter, a method of generating a target intraoral model in which scanned teeth are moved to a target position is described with reference to FIGS. 11 to 16.

FIG. 11 is a flowchart illustrating processes of a method of generating and displaying a target intraoral model in which scanned teeth are moved to a target position, according to one embodiment.

Referring to FIG. 11, in operation 1110, the data processing device 100 may determine a position of a planned prosthetic tooth for prosthetizing a missing tooth.

According to one embodiment, the data processing device 100 may determine central coordinates of a template tooth having a tooth number corresponding to a tooth number of a missing tooth as a position of a planned prosthetic tooth.

In operation 1120, the data processing device 100 may generate a planned prosthetic tooth by using at least one of a scanned tooth symmetrical to a tooth number of a missing tooth, a template tooth symmetrical thereto, and a template tooth corresponding to the tooth number of the missing tooth.

According to one embodiment, the data processing device 100 may generate a closed tooth of the scanned tooth based on a scanned tooth corresponding to a crown portion and a template tooth of a template tooth model. Although the closed tooth for a normal scanned tooth may be generated as described above, the planned prosthetic tooth does not include a scanned tooth corresponding to a corresponding tooth number, and accordingly, the data processing device 100 may generate the closed teeth by using the scanned tooth symmetrical to the tooth number of the missing tooth.

According to one embodiment, the data processing device 100 generates a planned prosthetic tooth by combining a scanned tooth symmetrical to the tooth number of the missing tooth with a template tooth symmetrical to the tooth number of the missing tooth and performing reflection processing on the combined closed tooth.

According to one embodiment, the data processing device 100 may generate a planned prosthetic tooth by performing reflection processing on a scanned tooth symmetrical to a tooth number of a missing tooth and combining the scanned tooth subjected to the reflection processing with a template tooth corresponding to the tooth number of the missing tooth.

In operation 1130, the data processing device 100 may arrange a closed teeth of a scanned teeth based on template tooth information of a template tooth model, and in this case, a planned prosthetic tooth may be arranged at the position of the planned prosthetic tooth determined in operation 1110.

In operation 1140, the data processing device 100 may align the closed teeth of the scanned teeth arranged based on a template tooth model with a customized curve generated based on the scanned teeth of a patient.

In operation 1150, the data processing device 100 may display, on a display, a target intraoral model including the closed teeth of the scanned teeth aligned with the customized curve.

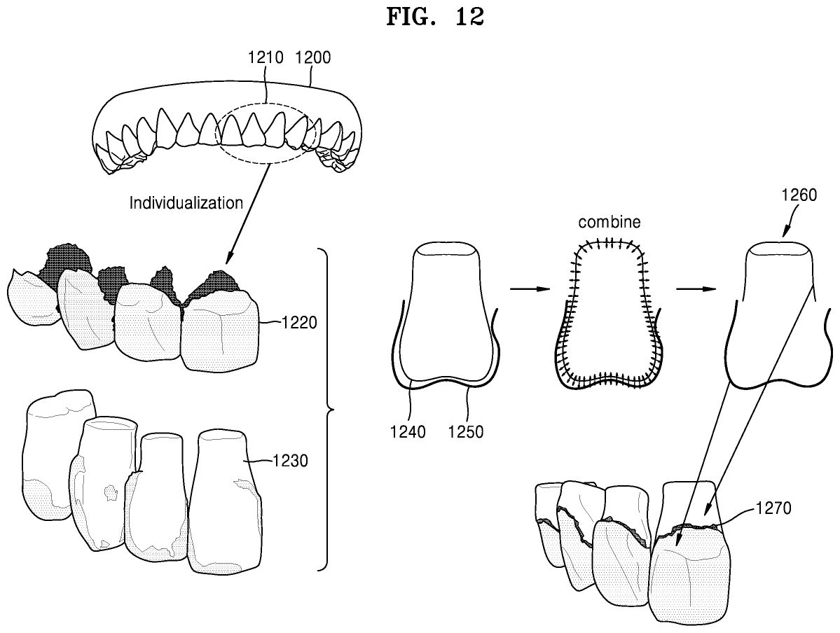

FIG. 12 is a reference view illustrating a method of generating closed teeth of scanned teeth, according to one embodiment.

The scan device 200 may obtain data on an object by scanning a surface of the object, and data on a dental root region of teeth 1210 covered by a gingiva 1200 may not be obtained. Accordingly, when teeth are individualized from an intraoral image, the individualized tooth cannot but include an open tooth 1220 without a dental root region of the tooth. When a tooth movement or an orthodontic simulation is performed based on an image without a dental root region, there is a problem in that an unnatural simulation may be provided. Therefore, the data processing device 100 may generate a tooth image 1240 including a dental root region of a tooth to provide a natural tooth image or simulation.

The data processing device 100 may align and combine the scanned tooth 1220 having only a crown portion with a template tooth 1230 of a template tooth model. When aligning the scanned tooth 1220 with the template tooth 1230 of the template tooth model, the data processing device 100 may use various alignment algorithms, and for example, an algorithm, such as the known ICP.

When the scanned tooth 1220 is aligned with the template tooth 1230, the data processing device 100 may combine the scanned tooth 1220 with the template tooth 1230 by performing a combination test. The combination test may be a nearest neighbor test or a ray intersection test. For example, the data processing device 100 may generate a composite image by performing the nearest neighboring test. Specifically, the nearest neighboring test means that a vertex of the scanned tooth 1240 in the closest distance from a certain vertex of the template tooth 1250 is found and a distance between the vertex of the template tooth 1250 and a vertex of the closest scanned tooth 1240 is tested. When the distance is equal to or less than a preset critical distance, the vertex of the template tooth 1250 is removed, and when the distance exceeds the preset critical distance, an image 1260 in which a scanned tooth is combined with a template tooth may be obtained by using the template tooth 1250. In this case, a non-smooth portion 1270 at a portion where a scanned tooth is combined with a template tooth may be generated as a natural composite image, that is, a closed tooth including a dental root region, through surface blending. The preset critical distance may be at least 1 mm and not more than 3 mm (for example, 2 mm), but is not limited thereto and may have various ranges. The nearest neighboring test may be at least one selected from K-d tree, Octree, and R-tree, but is not limited thereto. Also, the nearest neighboring test may be performed quickly after spatial decomposition is performed by using a spatial search algorithm, but is not limited thereto, and various algorithms may be used. A color of the generated closed tooth may be determined based on a color of the scanned tooth. For example, the data processing device 100 may determine a color value of a dental root region included in a composite image or a color value of surface data generated by surface blending by using a value obtained by averaging color values (for example, pixel values) of oral data included in scanned teeth. However, the embodiment is not limited thereto.

As described above, the data processing device 100 may generate a closed tooth of a scanned tooth including a dental root region by combining a scanned tooth corresponding to a crown portion with a template tooth.

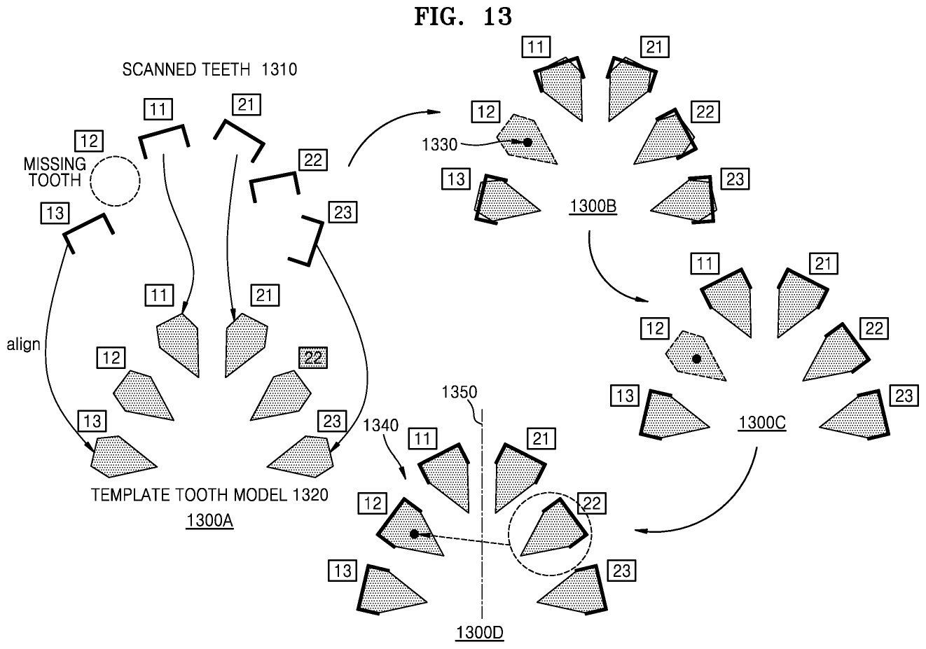

FIG. 13 is a reference diagram illustrating an example of a method of generating closed teeth of scanned teeth including a missing tooth and arranging the generated closed teeth in a template tooth model, according to one embodiment.

Referring to 1300A of FIG. 13, six teeth of the scanned teeth, that is, tooth numbers 11, 12, 13, 21, 22, and 23 are illustrated for the sake of convenience of description, and the tooth number 12 represents a missing tooth.

The data processing device 100 may align each scanned teeth of scanned teeth 1310 with a template tooth of a template tooth model having a corresponding tooth number. For example, the data processing device 100 may align a scanned tooth of the tooth number 11 with a template tooth of the tooth number 11, and align a scanned tooth of the tooth number 21 with a template tooth of the tooth number 21. Each template tooth of the template tooth model is data having a position by itself, and accordingly, by aligning the scanned teeth with the template teeth, the scanned teeth may be arranged at positions of the respective template teeth of the template tooth model. In this way, aligning the respective scanned teeth with the template teeth may include not only placing the scanned teeth at positions of the template teeth, but also correctly arranging the direction of each scanned tooth by aligning with reference to the template teeth. For example, when aligning the scanned tooth of the tooth number 11 with the template of the tooth number 11, rotate the scanned tooth of the tooth number 11 by a preset angle such that a direction of a tooth groove of the scanned tooth of the tooth number 11 coincides with a direction of a tooth groove of the template tooth of the tooth number 11, and the rotated scanned tooth of the tooth number 11 may be arranged at the position of template tooth of the tooth number 11. In this way, the scanned teeth are aligned at corresponding positions after being rotated with reference to the direction of the template teeth, the scanned teeth aligned with the template teeth may be obtained Aligning the scanned teeth based on the template teeth may be performed through, for example, the ICP algorithm described above.

In this way, when a scanned tooth is aligned with a template tooth having a corresponding tooth number, a form illustrated in 1300B may be obtained. All of the scanned teeth of tooth numbers 11, 13, 21, 22, and 23 may be aligned with the corresponding template teeth, and tooth number 12 corresponds to a missing tooth and there is no scanned tooth therefor, and accordingly, the scanned teeth are illustrated to be not aligned.

Then, as illustrated in 1300C, the data processing device 100 may generate a closed tooth including a composite image, that is, a dental root region, by combining each template tooth with aligned scanned tooth.

Referring to 1300D, in order to generate a planned prosthetic tooth for prosthetizing a missing tooth, the data processing device 100 may first obtain a closed tooth symmetrical to a tooth number of the missing tooth and perform reflection processing on the obtained closed tooth to arrange the closed tooth at a position of the planned prosthetic tooth. A closed tooth symmetrical to a tooth number of a missing tooth may be obtained by combining a scanned tooth symmetrical to the tooth number of the missing tooth with a template tooth symmetrical to the tooth number of the missing tooth.

The data processing device 100 may identify the tooth number 22 of the scanned tooth symmetrical to the tooth number 12 of the missing tooth based on a center line 1350 among the scanned teeth illustrated in 1300D. For example, the data processing device 100 may store in advance tooth numbers respectively symmetrical to the tooth numbers. For example, as illustrated in the second region 620 illustrated in FIG. 6, tooth number symmetry relationship information may be stored in a way in which the tooth number is incremented by 1 from the number 11 on the left side of the center in the upper jaw teeth, the tooth number is incremented by 1 from the number 21 on the right side of the center in the upper jaw teeth, the tooth number is incremented by 1 from the number 41 on the left side of the center in the lower jaw teeth, and the tooth number is incremented by 1 from the number 31 on the right side of the center in the lower jaw teeth. According to the symmetrical relationship, tooth numbers in a symmetrical relationship may be checked by identifying tooth numbers that differ only in a 10's digit and is the same in a 1's digit in a two-digit tooth number, among teeth symmetrical to certain teeth.

The data processing device 100 may combine a scanned tooth symmetrical to a tooth number of a missing tooth with a template tooth symmetrical to the tooth number of the missing tooth according to the method described with reference to FIG. 12, among the teeth found according to the symmetrical relationship.

Next, the data processing device 100 needs to arrange, in a symmetrical position, a closed teeth symmetrical to the tooth number of the missing tooth among the teeth generated by the combination, and accordingly, the teeth may be processed to be suitable for the orientation of a planned prosthetic tooth. For example, the data processing device 100 may use a reflection matrix such that the closed tooth symmetrical to the tooth number of the missing tooth is suitable for the orientation of the planned prosthetic tooth. Referring to 1300C, the planned prosthetic tooth is symmetrical to the closed tooth symmetrical to the tooth number of the missing tooth with respect to the center line 1350 as if reflected in a mirror, and accordingly, in order to arrange the closed tooth in a position of the planned prosthetic tooth, an orientation of the closed tooth needs to be adjusted to correspond to an orientation of the planned prosthetic tooth. Therefore, the data processing device 100 may obtain a planned prosthetic tooth by performing reflection processing on the closed tooth.

For example, a closed tooth symmetrical to the missing tooth of the tooth number 12, that is, a closed tooth of the tooth number 22 may be obtained by combining a scanned tooth of a tooth number 22 symmetrical to the tooth number 12 of the missing tooth with a template tooth of the tooth number 22 symmetrical to the tooth number 12 of the missing tooth. Then, the data processing device 100 may generate a planned prosthetic tooth by performing reflection processing on the closed tooth of the tooth number 22.

The closed tooth of the tooth number 22, which is subjected to reflection processing in this way, may be arranged at a position of the planned prosthetic tooth. That is, the data processing device 100 may arrange a planned prosthetic tooth 1340 such that a central position of the generated planned prosthetic tooth 1340 is located at a central position 1330 of the template tooth of the tooth number 12, which is a position of the planned prosthetic tooth.

In this way, the data processing device 100 may arrange scanned teeth including a missing tooth in a template tooth model.

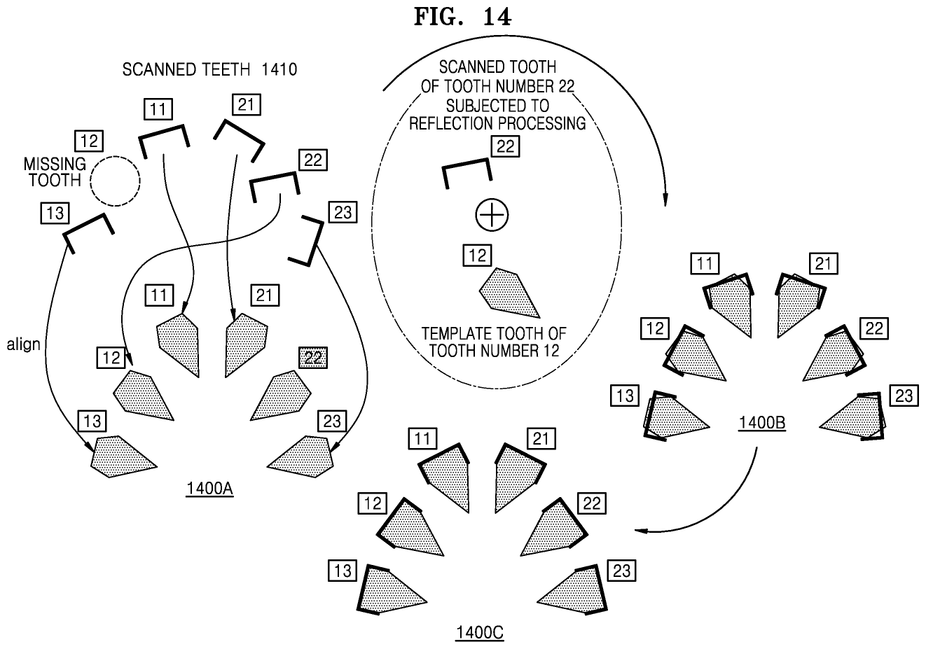

FIG. 14 is a reference diagram illustrating another example of a method of generating closed teeth of scanned teeth including a missing tooth and arranging the generated closed teeth in a template tooth model, according to one embodiment.

A difference between the method illustrated in FIG. 14 and the method illustrated in FIG. 13 is that, in FIG. 13, a planned prosthetic tooth is generated by combining a scanned tooth symmetrical to a tooth number of a missing tooth with a template tooth symmetrical to the tooth number of the missing tooth to generate a closed tooth and by performing reflection processing on the generated closed tooth, and in FIG. 14, a planned prosthetic tooth is generated by performing reflection processing on a scanned tooth symmetrical to a tooth number of a missing tooth and combining the scanned tooth subjected to the reflection processing with a template tooth corresponding to the tooth number of the missing tooth.

Referring to 1400A of FIG. 14, the data processing device 100 may align scanned teeth 1410 with template teeth of a template tooth model having tooth numbers respectively corresponding thereto. For example, the data processing device 100 may align a scanned tooth of a tooth number 11 with a template tooth of the tooth number 11, and align a scanned tooth of a tooth number 21 with a template tooth of the tooth number 21. Each template tooth of the template tooth model is data having a position by itself, and accordingly, by aligning the scanned teeth with the template teeth, the scanned teeth may be arranged at positions of the respective template teeth of the template tooth model.

In this case, the data processing device 100 may perform reflection processing on a scanned tooth of a tooth number 22 symmetrical to the tooth number 12 of a missing tooth, and aligns the scanned tooth subjected to the reflection processing with a template tooth of the tooth number 12 corresponding to the tooth number 12 of the missing tooth.

In this way, when a scanned tooth is aligned with a template tooth having a corresponding tooth number, the form illustrated in 1400B may be obtained. Scanned teeth of tooth numbers 11, 13, 21, 22, and 23 may be respectively aligned with corresponding template teeth, and a scanned tooth corresponding to the tooth number 12 may be subjected to reflection processing and may be aligned with a template tooth of the tooth number 12 corresponding to the tooth number 12 of a missing tooth.

Next, as illustrated in 1400C, the data processing device 100 may generate a composite image, that is, closed teeth including a dental root region, by combining the respective template teeth with scanned teeth aligned with the respective template teeth.

In this way, the data processing device 100 may arrange the scanned teeth including a missing tooth in a template tooth model.

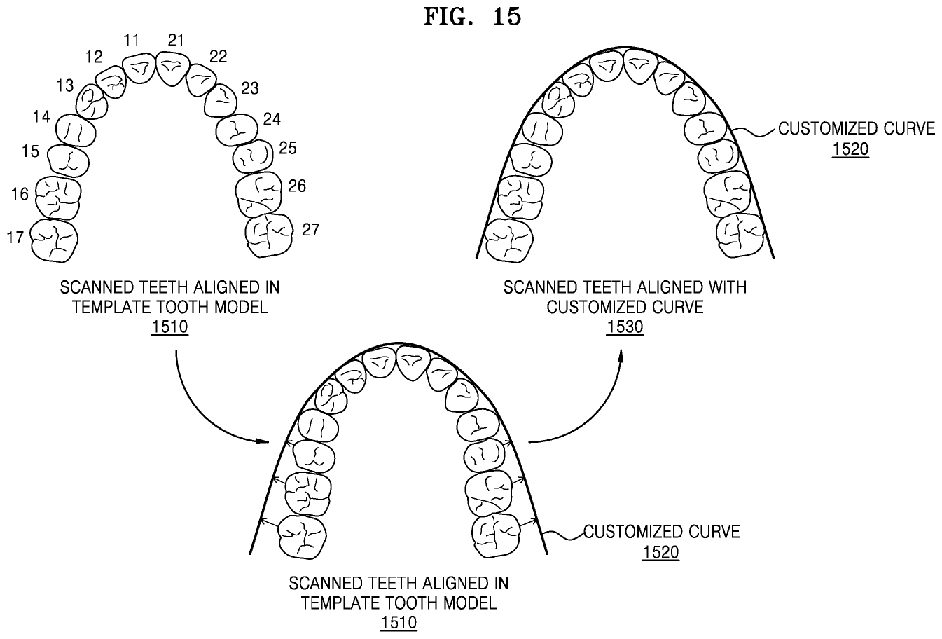

FIG. 15 is a reference view illustrating an example of a method of aligning scanned teeth aligned in a template tooth model with a customized curve, according to one embodiment.

Referring to FIG. 15, it is possible to obtain scanned teeth 1510 aligned in a template tooth model by aligning the scanned tooth including a missing tooth in a template tooth model according to the method illustrated in FIG. 13 or 14. However, the template tooth model may have a template curve of an ideal shape, and the template curve may differ greatly from an actual jaw arch state of a patient. Therefore, the scanned teeth 1510 aligned in a template tooth model are far from a target intraoral model after orthodontic treatment of a patient, and the orthodontic treatment of the patient may not be performed naturally. Therefore, it may be preferable that the data processing device 100 aligns the scanned teeth aligned in a template tooth model with a customized curve 1520 obtained by reflecting an oral cavity shape of a patient.

According to one embodiment, the data processing device 100 may generate a customized curve suitable for an oral cavity shape of a patient based on one or more scanned teeth among scanned teeth included in a three-dimensional intraoral model of the patient. Which tooth is used as a tooth based on generation of a customized curve, or the number of teeth based on generation of the customized curve may be determined in various ways.

According to one embodiment, the data processing device 100 may determine a tooth based on generation of a customized curve as a tooth having a small amount of movement among teeth. Specifically, a tooth having a small amount of movement does not move well because a dental root thereof is the deepest, and when a curve is generated based on the teeth, a line with an aesthetically pleasing curve shape may be derived.

According to one embodiment, the data processing device 100 may determine a tooth based on generation of a customized curve, and then determine which point of the determined tooth the customized curve has to pass through. Which part of a tooth a customized arch line has to pass through may be determined based on various characteristics of the tooth. Characteristics of tooth may include cusps, fossae, a ridge, distance-based metrics or shape-based metrics, and a bucal point. Because the scanned tooth 1510 aligned in a template tooth model are rotated with reference to template teeth of the template tooth model, when aligning the scanned teeth with a customized curve, positions of the teeth may be moved. In this case, by moving the scanned teeth by a preset distance in a horizontal direction or in a vertical direction, a preset point of each scanned tooth may be arranged to meet the customized curve 1520.

According to one embodiment, the data processing device 100 may obtain scanned teeth 1530 aligned with the customized curve 1520 by moving the scanned teeth 1510 aligned in a template tooth model to be aligned with the customized curve 1520 generated by reflecting an oral cavity shape of a patient. In this way, positions of the scanned teeth aligned with a customized curve may become target positions of the teeth of a patient after correction.

A final tooth position of a patient obtained according to the embodiments described above may be used for each operation of actual orthodontic treatment. Also, by showing the final positions of teeth of a patient obtained in this way to the patient, the patient may recognize the final positions of the teeth targeted for orthodontic treatment.

According to one embodiment, the data processing device 100 may display a screen indicating the final positions of teeth on a display to show how a state of a tooth of a patient changes according to an orthodontic plan.

According to one embodiment, the data processing device 100 may display a screen indicating a current tooth position and a final tooth position of a patient on a display to show how a state of a tooth of the patient changes according to an orthodontic plan.

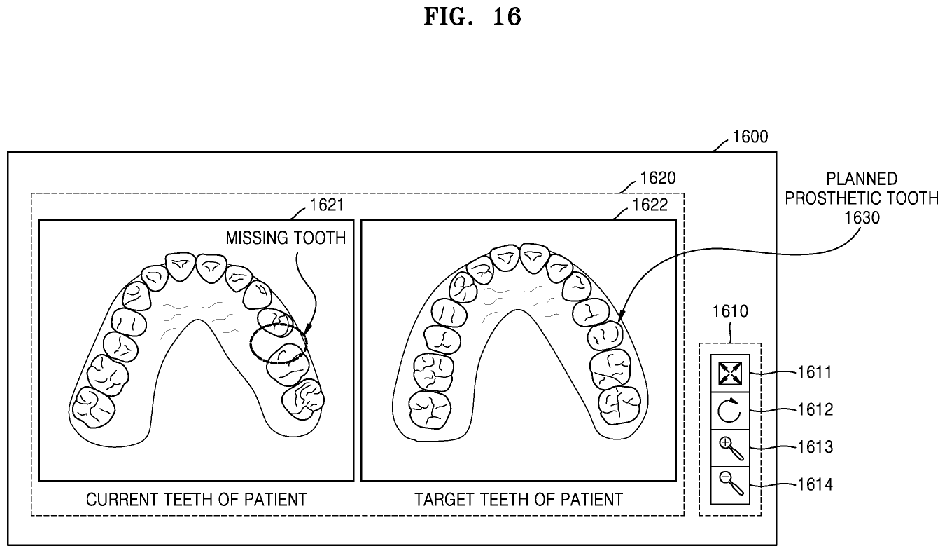

FIG. 16 illustrates an example of a graphical user interface illustrating a current tooth position and a final tooth position of a patient, according to one embodiment.

Referring to FIG. 16, the data processing device 100 may generate a user interface screen 1600 and output the user interface screen 1660 through a display. Here, the user interface screen 1600 may include one or more menu bars for allowing a user (for example, a dentist or so on) to use data obtained by scanning teeth by using an intraoral scanner.

The user interface screen 1600 may include a menu bar 1610 including at least one menu for editing or changing an obtained intraoral image. For example, the menu bar 1610 may include menus including a full screen view menu 1611, previous image view 1612, an intraoral image enlargement menu 1613, an intraoral image reduction menu 1614, and so on.

The user interface screen 1600 may include a window 1620 for displaying an orthodontic plan for a patient undergoing orthodontic treatment. A window 1620 may display together current teeth 1621 displayed by scanning the current teeth of a patient undergoing orthodontic treatment, and target teeth 1622 of teeth of the patient generated according to the description given with reference to FIGS. 1 to 15 based on current tooth positions of the patient, and accordingly, the patient may know positions of the current teeth after orthodontic treatment.

In particular, according to the embodiments of the present disclosure, even when the current teeth 1621 of a patient include a missing tooth 11, the data processing device 100 may display a state in which a planned prosthetic tooth 1630 for prosthetizing a missing tooth 11 is naturally arranged at a position of the planned prosthetic tooth when displaying states of target teeth 1622 of the patient. Also, although the planned prosthetic tooth 1630 is displayed, in the example illustrated in FIG. 16, in a color to be clearly displayed, the data processing device 100 may adjust and display transparency of the color of the planned prosthetic tooth 1630. Therefore, the data processing device 100 may clearly display a form and a shape of a planned prosthetic tooth by adjusting transparency of a color of the planned prosthetic tooth 1630 to a low level. Alternatively, the data processing device 100 may adjust the transparency of the color of the planned prosthetic tooth 1630 to a high level or the greatest value to display only the space where the planned prosthetic tooth is arranged even when the planned prosthetic tooth is not displayed and is in the same state as a current tooth state of a patient.

A three-dimensional intraoral model processing method according to one embodiment of the present disclosure may be implemented in the form of program instructions that may be executed through various computes and may be recorded on a computer-readable medium. Also, an embodiment of the present disclosure may be a computer-readable storage medium in which one or more programs including at least one instruction for performing an intraoral image processing method is recorded.

The computer-readable storage medium may include program instructions, data files, data structures, and so on alone or in combination. Here, the computer-readable storage medium may include a magnetic medium, such as a hard disk, a floppy disk, on a magnetic tape, an optical medium, such as compact disk (CD)-ROM or a digital video disk (DVD), a magneto-optical medium, such as a floptical disk, and a hardware device configured to store and execute program instructions, such as ROM, random access memory (RAM), or flash memory.

Here, the machine-readable storage medium may be provided in the form of a non-transitory storage medium. Here, the ‘non-transitory storage medium’ may indicate that a storage medium is a tangible device. Also, the ‘non-transitory storage medium’ may include a buffer in which data is temporarily stored.

According to one embodiment, the intraoral image processing method according to various embodiments disclosed in the present specification may be included in a computer program product. A computer program product may be distributed in the form of a machine-readable storage medium (for example, compact disc read only memory (CD-ROM)). Alternatively, the computer program product may be directly distributed (for example, downloaded or uploaded) online through an application store (for example, play store or so on) or between two user devices (for example, smartphones). Specifically, the computer program product according to the embodiment may include a storage medium in which a program including at least one instruction is recorded to perform the intraoral image processing method according to the embodiment.

Although embodiments are described in detail above, the scope of the present disclosure is not limited thereto, and various modifications and improvements of those skilled in the art using the basic concept of the present disclosure defined in the following claims are also within the scope of the present disclosure.

1. A three-dimensional intraoral model processing device comprising:
a memory storing one or more instructions; and
a processor, by executing the one or more instructions, being configured to:
identify a missing tooth in scanned teeth of a three-dimensional intraoral model obtained by scanning teeth,
generate a planned prosthetic tooth for prosthetizing the missing tooth, by using one of the scanned teeth of the three-dimensional intraoral model,
move the scanned teeth including the generated planned prosthetic tooth to a final target position, and
display a target intraoral model including the scanned teeth moved to the final target position.

2. The three-dimensional intraoral model processing device of claim 1, wherein the processor, by executing the one or more instructions, is configured to individualize the scanned teeth of the three-dimensional intraoral model, and assign a tooth number to each of the individualized scanned teeth, wherein a tooth number that is not assigned to a scanned tooth is identified as a tooth number of the missing tooth.

3. The three-dimensional intraoral model processing device of claim 1, wherein the processor, by executing the one or more instructions, is configured to generate the planned prosthetic tooth for prosthetizing the missing tooth, according to a user input for selecting the tooth number of the missing tooth.

4. The three-dimensional intraoral model processing device of claim 1, wherein the processor, by executing the one or more instructions, is configured to generate closed teeth by combining each of the scanned teeth with a dental root of a template tooth of a corresponding tooth number, wherein a closed tooth corresponding to the planned prosthetic tooth is generated by using a scanned tooth symmetrical to the missing tooth.

5. The three-dimensional intraoral model processing device of claim 4, wherein the processor, by executing the one or more instructions, is configured to identify a position of a template tooth corresponding to a tooth number of the missing tooth, and generate the closed tooth corresponding to the planned prosthetic tooth by symmetrically moving and arranging a closed tooth symmetrical to the template tooth corresponding to the tooth number of the missing tooth to a position of the template tooth corresponding to the tooth number of the missing tooth.

6. The three-dimensional intraoral model processing device of claim 4, wherein the processor, by executing the one or more instructions, is configured to identify a position of a template tooth corresponding to a tooth number of the missing tooth, and generate the closed tooth corresponding to the planned prosthetic tooth by symmetrically moving the scanned tooth symmetrical to the missing tooth and combining the scanned tooth symmetrical to the missing tooth with a dental root of the template tooth corresponding to the tooth number of the missing tooth.

7. The three-dimensional intraoral model processing device of claim 4, wherein the processor, by executing the one or more instructions, is configured to obtain the scanned teeth moved to the final target position, by aligning the closed teeth with a customized curve generated based on the scanned teeth.

8. The three-dimensional intraoral model processing device of claim 4, wherein the processor, by executing the one or more instructions, is configured to control transparency of a color of the closed tooth generated in correspondence with the tooth number of the missing tooth, to be adjustably displayed.

9. The three-dimensional intraoral model processing device of claim 1, wherein
the processor, by executing the one or more instructions, is configured to output a user interface including one or more items selectable by a user, according to a user input for selecting a tooth number recognized as the missing tooth, and
the one or more items include at least one of a first item selectable to recognize a scanned tooth corresponding to a tooth number recognized as the missing tooth among the scanned teeth of the three-dimensional intraoral model, or a second item selectable to provide a planned prosthetic tooth for prosthetizing the recognized missing tooth.

10. The three-dimensional intraoral model processing device of claim 9, wherein the processor, by executing the one or more instructions, is configured to receive a user input designating a scanned tooth corresponding to the missing tooth, according to a user input for selecting the first item, assign a tooth number to the designated scanned tooth, and output the assigned tooth number.

11. An operating method of a three-dimensional intraoral model processing device, the operating method comprising:
identifying a missing tooth among scanned teeth of a three-dimensional intraoral model obtained by scanning teeth;
generating a planned prosthetic tooth for prosthetizing the missing tooth, by using one of the scanned teeth of the three-dimensional intraoral model;
moving the scanned teeth including the generated planned prosthetic tooth to a final target position; and
displaying a target intraoral model including the scanned teeth moved to the final target position.

12. The operating method of claim 11, further comprising individualizing the scanned teeth of the three-dimensional intraoral model, and assigning a tooth number to each of the individualized scanned teeth, wherein a tooth number that is not assigned to the scanned teeth is identified as a tooth number of the missing tooth.

13. The operating method of claim 12, further comprising generating closed teeth by combining each of the scanned teeth with a dental root of a template tooth of a corresponding tooth number, wherein a closed tooth corresponding to the tooth number of the missing tooth is generated by using a scanned tooth symmetrical to the missing tooth.

14. The operating method of claim 13, further comprising:
identifying a position of a template tooth corresponding to the tooth number of the missing tooth; and
generating a closed tooth corresponding to the missing tooth by symmetrically moving and arranging a closed tooth symmetrical to the template tooth corresponding to the tooth number of the missing tooth to the position of the template tooth corresponding to the tooth number of the missing tooth.

15. The operating method of claim 13, further comprising:
identifying a position of a template tooth corresponding to the tooth number of the missing tooth; and
generating a closed tooth corresponding to the missing tooth by symmetrically moving the scanned tooth symmetrical to the missing tooth and combining the scanned tooth symmetrical to the missing tooth with the dental root of the template tooth corresponding to the tooth number of the missing tooth.

16. The operating method of claim 13, further comprising obtaining the scanned teeth moved to the final target position, by aligning the closed teeth with a customized curve generated based on the scanned teeth.

17. The operating method of claim 13, further comprising controlling transparency of a color of the closed tooth generated in correspondence with the tooth number of the missing tooth, to be adjustably displayed.

18. The operating method of claim 11, further comprising outputting a user interface including one or more items selectable by a user, according to a user input for selecting a tooth number recognized as the missing tooth,
wherein the one or more items include at least one of a first item selectable to recognize a scanned tooth corresponding to a tooth number recognized as the missing tooth among the scanned teeth of the three-dimensional intraoral model, or a second item selectable to provide a planned prosthetic tooth for prosthetizing the recognized missing tooth.

19. The three-dimensional intraoral model processing method of claim 18, further comprising receiving a user input designating a scanned tooth corresponding to the missing tooth, according to a user input for selecting the first item, assigning a tooth number to the designated scanned tooth, and outputting the assigned tooth number.

20. A non-transitory computer-readable storage medium in which a program including at least one instruction is recorded to perform a three-dimensional intraoral model processing method by using a computer, the three-dimensional intraoral model processing method comprising:
identifying a missing tooth among scanned teeth of a three-dimensional intraoral model obtained by scanning teeth;
generating a planned prosthetic tooth for prosthetizing the missing tooth, by using one of the scanned teeth of the three-dimensional intraoral model;
moving the scanned teeth including the generated planned prosthetic tooth to a final target position; and
displaying a target intraoral model including the scanned teeth moved to the final target position.
